(19) United States
(12) Patent Application Publication
(10) Pub. No.: US 2026/0216038 A1
(43) Pub. Date: Jul. 30, 2026

(54) ARGIRELINE CO-ASSEMBLED SYSTEM, PREPARATION METHOD THEREFOR, USE THEREOF, AND SKIN CARE COMPOSITION COMPRISING THEREOF

(71) Applicant: THE PROCTER & GAMBLE COMPANY, Cincinnati, OH (US)

(72) Inventors: Lu ZHANG, Singapore (SG); Jiaheng ZHANG, Shenzhen (CN); Zhenyuan WANG, Shenzhen (CN); Biao WANG, Shenzhen (CN); Yuxiang ZHU, Shenzhen (CN)

(21) Appl. No.: 19/457,338

(22) Filed: Jan. 23, 2026

(30) Foreign Application Priority Data
Jan. 24, 2025 (WO) PCT/CN2025/074593
Feb. 27, 2025 (WO) PCT/CN2025/079576

(51) Int. Cl. A61K 8/64; A61K 8/365; A61K 8/44; A61Q 19/08

(52) U.S. Cl. CPC A61K 8/64; A61K 8/365; A61K 8/44; A61Q 19/08

(57) ABSTRACT

An argireline co-assembled system formed from betaine-malic acid as a deep eutectic solvent and argireline through non-covalent interaction and a method for preparing an argireline co-assembled system. The system can provide improved skin permeability of argireline and improved stability of argireline in skin care compositions.

### FIELD

The present disclosure is directed to an argireline co-assembled system formed from betaine-malic acid as a deep eutectic solvent and argireline through non-covalent interaction. The present disclosure is also directed to a preparation method of such argireline co-assembled system, a use thereof, and a skin care composition comprising thereof. The present disclosure provides improved skin permeability of argireline and improved stability of argireline in skin care compositions.

### BACKGROUND

Skin is the first line of defense against environmental insults that would otherwise damage sensitive underlying tissue and organs. Additionally, skin plays a key role in a person's physical appearance. Not surprisingly, most people would like to have heathy, younger looking skin. Unfortunately for some people, the tell-tale signs of aging such as thinning skin, wrinkles, and age spots are an undesirable reminder of the disappearance of youth. The desire for healthy, younger looking skin has led to the development of numerous skin care products marketed to treat the various skin conditions associated with aging and unhealthy skin. These skin care products typically include one or more active ingredients for treating a skin condition of interest.

Peptides and peptide derivatives are commonly used in skin care products as active ingredients. For example, argireline, also known as acetyl hexapeptide-8, is a neurotransmitter inhibitory peptide consisting of six amino acids, and is an anti-wrinkle active polypeptide. It mimics the N-terminal structure of the SNAP-25 protein in the SNARE complex, and can competes with SNAP-25 for the position in the complex, thereby affecting the formation and stability of the SNARE complex, such that a vesicle is unable to efficiently bind to a membrane to release acetylcholine. As a result, the muscular electrical pulse signals are blocked, the release of neurotransmitters is inhibited, thereby reducing the formation of expression wrinkles and dynamic wrinkles. Argireline is recognized in the cosmetic field in terms of safety and effectiveness and is widely used.

Although argireline has many advantages as an effective anti-wrinkle ingredient in cosmetics, it has the following limitations (i) and (ii) in use:
(i) Skin permeability problem: This is due to the high molecular weight and hydrophilicity of argireline and the ionizable groups in its structure, which make its skin delivery extremely challenging; and
(ii) Stability and formulation compatibility: In cosmetic formulations, argireline needs to remain stable and be compatible with other ingredients. To achieve this, special formulation techniques or additives may be required. The anti-wrinkle effect of argireline may not be as pronounced or long-lasting as that of injectable creotoxin, and long-term and continuous use is required to maintain the effect.

Therefore, there is a need to improve skin permeability of argireline and improved stability of argireline in skin care compositions.

### SUMMARY

Disclosed herein is an argireline co-assembled system, wherein the argireline co-assembled system is a co-assembled system formed from betaine-malic acid as a deep eutectic solvent and argireline through non-covalent interaction.

Preferably, the mass ratio of argireline to betaine-malic acid deep eutectic solvent is 1:5 to 1:100. Preferably, the molar quantity ratio of betaine to malic acid in the deep eutectic solvent is 1:5 to 5:1.

Also disclosed herein is a method for preparing an argireline co-assembled system, the method comprising the steps of:
(i) preparing a deep eutectic solvent, the deep eutectic solvent being betaine-malic acid;
(ii) mixing argireline and the betaine-malic acid deep eutectic solvent and stirring at a temperature of from about 20° C. to about 40° C.; and
(iii) performing ultrasonication to induce co-assembling of the argireline and the betaine-malic acid deep eutectic solvent, thereby obtaining the argireline co-assembled system.

Preferably, the mass ratio of argireline to betaine-malic acid deep eutectic solvent is 1:5 to 1:100. Preferably, the molar quantity ratio of betaine to malic acid in the deep eutectic solvent is 1:5 to 5:1.

Preferably, the above step (ii) mixing and stirring argireline and the betaine-malic acid deep eutectic solvent comprises the sub-steps of:
(ii-a) mixing water and the betaine-malic acid deep eutectic solvent, stirring at a temperature of from about 20° C. to about 40° C. and homogenizing at a pressure of from about 2000 bar to about 8000 bar, such that the betaine-malic acid deep eutectic solvent is uniformly dispersed;
(ii-b) mixing an argireline aqueous solution and the above uniform dispersion of betaine-malic acid deep eutectic solvent at a temperature of from about 20° C. to about 40° C.

Preferably, in the above step (ii) and its sub-steps:
the stirring time is from about 1 h to about 12 h, and the stirring rotation speed is from about 300 rpm to about 600 rpm;
the homogenization time is from about 20 min to about 60 min; and

Preferably, in the above step (iii), the ultrasonication treatment involves: ultrasonicating at a power of from about 100 W to about 600 W and a frequency of from about 10 kHz to about 50 kHz for about 2 s and pausing for about 2 s, for a duration of from about 1 h to about 4 h.

Preferably, the above step (i) the preparation of the betaine-malic acid deep eutectic solvent comprises the sub-steps of:
(i-a) under the protection of an inert gas, mixing betaine, malic acid, and an appropriate amount of water, stirring under heating,
(i-b) performing ultrasonication treatment, and
(i-c) performing rotary evaporation to remove water, thereby obtaining the betaine-malic acid deep eutectic solvent.

Preferably, in the above step (i) and its sub-steps:
the inert gas is nitrogen, and the pressure of the nitrogen is 0.1 MPa to 20 MPa;
the stirring temperature is from about 30° C. to about 80° C., the stirring time is from about 1 h to about 48 h, and the stirring rotation speed is from about 300 rpm to about 600 rpm;
the ultrasonication treatment involves: ultrasonicating at a power of from about 100 W to about 600 W and a frequency of from about 10 kHz to about 50 kHz for about 2 s and pausing for about 2 s, for a duration of from about 1 h to about 4 h; and
the temperature of the rotary evaporation is from about 60° C. to about 80° C.

The present disclosure is also directed to the use of such argireline co-assembled system for anti-wrinkle benefit.

The present disclosure is further directed to a skin care composition comprising an effective amount of such argireline co-assembled system.

In summary, the present disclosure is directed to an argireline co-assembled system formed from betaine-malic acid as a deep eutectic solvent and argireline through non-covalent interaction. The present disclosure is also directed to a preparation method of such argireline co-assembled system, a use thereof, and a skin care composition comprising thereof.

The present disclosure provides improved skin permeability of argireline and improved stability of argireline in skin care compositions.

Argireline has the problems of poor skin permeability and poor stability during its use. In contrast to the prior art, in the present disclosure, a betaine-malic acid deep eutectic solvent is screened and synthesized, and then the deep eutectic solvent is combined with argireline to construct together a co-assembled system through non-covalent interaction, i.e., weak interactions such as hydrogen bonding, van der Waals interaction, and electrostatic interaction. Besides being able to work synergistically with argireline to prevent wrinkles, malic acid can also act as a component of the deep eutectic solvent to increase the solubility of argireline, improve the oil-water distribution coefficient of argireline, and at the same time promote the transdermal absorption of argireline by means such as reducing permeation resistance. The co-assembled system can also reduce the degree of freedom of the molecular movement of argireline, reduce its contact area, reduce the risk of its enzymatic degradation during transdermal process, and significantly improve its stability, thereby improving its bioavailability and solving the problem of instability of argireline, which lays a foundation for the use of argireline in pharmaceutical preparations.

A deep eutectic solvent (DES for short) refers to a two- or three-component deep eutectic mixture composed of a hydrogen bond acceptor (such as a quaternary ammonium salt) and a hydrogen bond donor (such compound as an amide, a carboxylic acid, a polyol, etc.) that are combined in a certain stoichiometric ratio. Its solidifying point is significantly lower than the melting point of the pure substance of each component. It is a novel green supramolecular solvent. The deep eutectic solvent has characteristics such as low melting point, low cost, low toxicity, easy preparation, reproducibility, biodegradability, and also has the characteristics of structural designability and adjustability that general solvents do not have. It has been widely used in the biopharmaceutical field, particularly transdermal delivery. The deep eutectic solvent may promote transdermal absorption of active molecules by means such as increasing the solubility of the active molecules, improving the oil-water distribution coefficient of the active molecules, and at the same time reducing the permeation resistance.

Malic acid is an alpha hydroxy acid (AHA), which has a different molecular action mechanism from that of argireline, and exerts an anti-wrinkle effect on a different aspect of skin aging. Malic acid reduces the appearance of fine lines and wrinkles by promoting the renewal of epidermal cells and enhancing the adhesion between skin cells. Its effect mainly manifests in promoting the synthesis of collagen and regulating skin pH, thereby maintaining the barrier function of the skin. This mechanism of action is distinct from the anti-wrinkle mechanism of argireline, suggesting that they are complementary, rather than competitive, in anti-wrinkle treatment.

Therefore, the use of malic acid in combination with argireline can achieve multi-directional intervention in the skin aging process, thereby enhancing the anti-wrinkle effect, without causing mutual cancellation of their effects. In addition, the exfoliating property of malic acid affects the skin barrier to some extent. While malic acid and argireline work synergistically against wrinkles, there is also the possibility of increasing the transdermal permeation of argireline, which further increases the bioavailability of argireline.

In the present disclosure, malic acid is used as a substrate, a latent ligand is screened, a deep eutectic solvent is synthesized, and then the deep eutectic solvent is combined with argireline to construct together a co-assembled system through non-covalent interaction, i.e., weak interactions such as hydrogen bond, van der Waals interaction, and electrostatic interaction. The introduction of the deep eutectic solvent increases the solubility of argireline, improves the oil-water distribution coefficient of argireline, and at the same time promotes the transdermal absorption of argireline by means such as reducing permeation resistance. The co-assembled system can also reduce the degree of freedom of the molecular movement of argireline, reduce its contact area, reduce the risk of its enzymatic degradation during transdermal process, and significantly improve its stability, thereby improving its bioavailability, which lays a foundation for the use of argireline in pharmaceutical preparations.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a schematic flow diagram illustrating the preparation of an argireline co-assembled system;

FIG. 2 is a schematic representation of the result of molecular docking in an argireline co-assembled system;

FIG. 3 is a schematic representation of the electrostatic potential distribution in an argireline co-assembled system;

FIG. 4 is a schematic representation of non-covalent interaction, i.e., weak interactions in an argireline co-assembled system;

FIG. 5 is a nuclear magnetic resonance hydrogen spectrogram of an argireline co-assembled system;

FIG. 6 is a two-dimensional nuclear magnetic resonance hydrogen spectrogram of an argireline co-assembled system;

FIG. 7 shows the result of a transmission electron microscopy test of an argireline aqueous solution;

FIG. 8 shows the result of a transmission electron microscopy test of an argireline co-assembled system;

FIG. 9 is a diagram showing the trend of relative viability of human skin fibroblasts under the action of different concentrations of an argireline co-assembled system;

FIG. 10 is diagram comparing the relative amounts of human collagen type I under the action of different samples;

FIG. 11 is a diagram comparing the relative amounts of MMP-1 under the action of different samples;

FIG. 12 is a diagram comparing the relative amounts of MMP-3 under the action of different samples; and

FIG. 13 is a statistical diagram showing the cumulative permeation amount per unit area, in 24 h, of an argireline co-assembled system and of an argireline aqueous solution in an in vitro transdermal diffusion experiment.

### DETAILED DESCRIPTION

Reference herein to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions onto a human skin surface such as the epidermis.

“Cosmetic composition” means a composition comprising a cosmetic agent and intended for non-therapeutic (i.e., medical) use. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Argireline Co-Assembled System and a Preparation Method Thereof

Argireline is a neurotransmitter inhibitory peptide consisting of six amino acids, and is an anti-wrinkle active polypeptide. It mimics the N-terminal structure of the SNAP-25 protein in the SNARE complex, and can competes with SNAP-25 for the position in the complex, thereby affecting the formation and stability of the SNARE complex, such that a vesicle is unable to efficiently bind to a membrane to release acetylcholine. As a result, the muscular electrical pulse signals are blocked, the release of neurotransmitters is inhibited, thereby reducing the formation of expression wrinkles and dynamic wrinkles. Argireline is recognized in the cosmetic field in terms of safety and effectiveness and is widely used. Despite of its many advantages, argireline also has the problems of poor skin permeability and poor stability, which limits its use in the cosmetic industry to a great extent.

On that basis, an embodiment of the present disclosure provides an argireline co-assembled system, aiming to solve the problems of poor skin permeability and instability of argireline. The argireline co-assembled system is a co-assembled system formed from betaine-malic acid as a deep eutectic solvent and argireline through non-covalent interaction, i.e., weak interactions such as hydrogen bonding, van der Waals interaction, and electrostatic interaction under specific conditions.

The argireline co-assembled system of the present disclosure is a co-assembled system formed from betaine-malic acid as a deep eutectic solvent and argireline through non-covalent interaction.

This embodiment provides the argireline co-assembled system by screening and synthesizing the betaine-malic acid deep eutectic solvent and then combining the deep eutectic solvent with argireline to construct together a co-assembled system through non-covalent interaction, i.e., weak interactions such as hydrogen bond, van der Waals interaction, and electrostatic interaction. Besides being able to work synergistically with argireline to prevent wrinkles, malic acid can also act as a component of the deep eutectic solvent to increase the solubility of argireline, improves the oil-water distribution coefficient of argireline, and at the same time promotes the transdermal absorption of argireline by means such as reducing permeation resistance. The co-assembled system can also reduce the degree of freedom of the molecular movement of argireline, reduce its contact area, reduce the risk of its enzymatic degradation during transdermal process, and significantly improve its stability, thereby improving its bioavailability and solving the problem of instability of argireline, which lays a foundation for the use of argireline in pharmaceutical preparations.

In this embodiment, malic acid is used as a target substrate, and a potential ligand capable of forming a deep eutectic solvent with malic acid is preliminarily screened, such as an organic base, including matrine, betaine, L-carnitine etc.; a polyhydric alcohol, including glycerin, propylene glycol, butylene glycol, ethoxydiglycol, etc. One or more of the above compounds are selected as the potential ligand and are used to form the deep eutectic solvent together with malic acid.

In this embodiment, the argireline co-assembled system is formed from argireline, the betaine-malic acid deep eutectic solvent, and water together through non-covalent interaction, i.e., weak interactions such as hydrogen bonding, van der Waals interaction, and electrostatic interaction. The co-assembled system can increase the solubility of argireline, improve the oil-water distribution coefficient of argireline, and at the same time promote the transdermal absorption of argireline by means such as reducing permeation resistance. The co-assembled system can also reduce the degree of freedom of the molecular movement of argireline, reduce its contact area, reduce the risk of its enzymatic degradation during transdermal process, and significantly improve its stability.

In one embodiment, the mass ratio of argireline to betaine-malic acid deep eutectic solvent is 1:5 to 1:100, further preferably 1:10 to 1:30; for example, the mass ratio may be 1:5, 1:10, 1:15, 1:20, 1:25, 1:30, 1:35, 1:40, 1:45, 1:50, 1:60, 1:70, 1:80, 1:90, 1:100, etc.

In one embodiment, the molar quantity ratio of betaine to malic acid in the deep eutectic solvent is 1:5 to 5:1, further preferably 1:3 to 3:1; for example, the molar quantity ratio may be 1:5, 1:4, 1:3, 1:2, 1:1, 2:1, 3:1, 4:1, 5:1, etc.

### Method of Preparing the Argireline Co-Assembled System

The present disclosure is also directed to a method for preparing an argireline co-assembled system, the method comprising the steps of
(i) preparing a deep eutectic solvent, the deep eutectic solvent being betaine-malic acid;
(ii) mixing argireline and the betaine-malic acid deep eutectic solvent and stirring at a temperature of from about 20° C. to about 40° C., preferably at a normal pressure; and
(iii) performing ultrasonication to induce co-assembling of the argireline and the betaine-malic acid deep eutectic solvent, thereby obtaining the argireline co-assembled system, wherein the ultrasonication is preferably performed at a room temperature.

This embodiments provides an argireline co-assembled system, which is a colorless transparent liquid. Argireline has the problems of poor skin permeability and poor stability during its use. The co-assembled system can increase the solubility of argireline, improve the oil-water distribution coefficient of argireline, and at the same time promote the transdermal absorption of argireline by means such as reducing permeation resistance. The co-assembled system can also reduce the degree of freedom of the molecular movement of argireline, reduce its contact area, reduce the risk of its enzymatic degradation during transdermal process, and significantly improve its stability.

In one embodiment, the mass ratio of argireline to betaine-malic acid deep eutectic solvent is 1:5 to 1:100, further preferably 1:10 to 1:30; for example, the mass ratio may be 1:5, 1:10, 1:15, 1:20, 1:25, 1:30, 1:35, 1:40, 1:45, 1:50, 1:60, 1:70, 1:80, 1:90, 1:100, etc.

In one embodiment, the molar quantity ratio of betaine to malic acid in the deep eutectic solvent is 1:5 to 5:1, further preferably 1:3 to 3:1; for example, the molar quantity ratio may be 1:5, 1:4, 1:3, 1:2, 1:1, 2:1, 3:1, 4:1, 5:1, etc.

Preferably, the above step (ii) mixing and stirring argireline and the betaine-malic acid deep eutectic solvent comprises the sub-steps of
(ii-a) mixing water and the betaine-malic acid deep eutectic solvent, stirring at a temperature of from about 20° C. to about 40° C. and homogenizing at a pressure of from about 2000 bar to about 8000 bar, such that the betaine-malic acid deep eutectic solvent is uniformly dispersed; and
(ii-b) mixing an argireline aqueous solution and the above uniform dispersion of betaine-malic acid deep eutectic solvent at a temperature of from about 20° C. to about 40° C.

Preferably, in the above step (ii) and its sub-steps, the stirring time is from about 1 h to about 12 h, and the stirring rotation speed is from about 300 rpm to about 600 rpm. In one embodiment, the temperature of the stirring is 20° C. to 40° C., further preferably 25° C. to 35° C.; for example, the temperature may be 20° C., 25° C., 30° C., 35° C., 40° C., etc.

In one embodiment, the time of the stirring is 1 h to 12 h, further preferably 2 h to 4 h; for example, the time of the stirring may be 1 h, 2 h, 3 h, 4 h, 5 h, 6 h, 7 h, 8 h, 9 h, 10 h, 11 h, 12 h, etc.

Preferably, in the above step (ii) and its sub-steps, the homogenization time is from about 20 min to about 60 min. In one embodiment, the time of the homogenizing is 20 min to 60 min, further preferably 35 min to 45 min; for example, the time may be 20 min, 25 min, 30 min, 35 min, 40 min, 45 min, 50 min, 55 min, 60 min, etc. In one embodiment, the pressure of the homogenizing at high pressure is 2000 bar to 8000 bar, further preferably 4000 bar to 6000 bar; for example, the pressure may be 2000 bar, 2500 bar, 3000 bar, 3500 bar, 4000 bar, 4500 bar, 5000 bar, 5500 bar, 6000 bar, 6500 bar, 7000 bar, 7500 bar, 8000 bar, etc.

Preferably, in the above step (iii), the ultrasonication treatment involves: ultrasonicating at a power of from about 100 W to about 600 W and a frequency of from about 10 kHz to about 50 kHz for about 2 s and pausing for about 2 s, for a duration of from about 1 h to about 4 h, so as to have an adequate intermolecular interaction forces of the components. In one embodiment, the ultrasonication power of the ultrasonication treatment is 20 W to 3000 W, further preferably 100 W to 1000 W; for example, the ultrasonication power may be 20 W, 50 W, 100 W, 150 W, 200 W, 300 W, 400 W, 500 W, 1000 W, 2000 W, 3000 W, etc. In one embodiment, the ultrasonication frequency of the ultrasonication treatment is 5 kHz to 100 kHz, further preferably 10 kHz to 50 kHz; for example, the ultrasonication frequency may be 5 kHz, 10 kHz, 20 kHz, 30 kHz, 40 kHz, 50 kHz, 60 kHz, 70 kHz, 80 kHz, 90 kHz, 100 kHz, etc. In one embodiment, the ultrasonication time of the ultrasonication treatment is 1 h to 4 h; for example, the ultrasonication time may be 1 h, 2 h, 3 h, 4 h, etc.

Preferably, the above step (i) the preparation of the betaine-malic acid deep eutectic solvent comprises the sub-steps of:
(i-a) under the protection of an inert gas, mixing betaine, malic acid, and an appropriate amount of water, stirring under heating,
(i-b) performing ultrasonication treatment, wherein the ultrasonication is preferably performed at a room temperature; and
(i-c) performing rotary evaporation to remove water, thereby obtaining the betaine-malic acid deep eutectic solvent.

Preferably, in the above step (i) and its sub-steps, the inert gas is nitrogen, and the pressure of the nitrogen is 0.1 MPa to 20 MPa. In one embodiment, the inert gas is nitrogen, and the pressure of the nitrogen is 0.1 MPa to 20 MPa, further preferably 10 MPa to 15 MPa. For example, the pressure may be 0.1 MPa, 0.5 MPa, 1 MPa, 2 MPa, 3 MPa, 4 MPa, 5 MPa, 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, etc.

Preferably, in the above step (i) and its sub-steps, the stirring temperature is from about 30° C. to about 80° C., the stirring time is from about 1 h to about 48 h, and the stirring rotation speed is from about 300 rpm to about 600 rpm. In one embodiment, the temperature of the stirring under heating is 30° C. to 80° C., more preferably 50° C. to 70° C.; for example, the temperature may be 30° C., 35° C., 40° C., 45° C., 50° C., 55° C., 60° C., 65° C., 70° C., 75° C., 80° C., etc. In one embodiment, the time of the stirring under heating is 1 h to 48 h, further preferably 2 h to 6 h; for example, the time of the stirring may be 2 h, 4 h, 6 h, 8 h, 10 h, 12 h, 14 h, 16 h, 18 h, 20 h, 22 h, 24 h, 26 h, 28 h, 30 h, 32 h, 34 h, 36 h, 38 h, 40 h, 42 h, 44 h, 46 h, 48 h, etc. In one embodiment, the rotational speed of the stirring is 100 rpm to 600 rpm, more preferably 300 rpm to 600 rpm; for example, the stirring rotation speed may be 100 rpm, 200 rpm, 300 rpm, 400 rpm, 500 rpm, 600 rpm, etc.

Preferably, in the above step (i) and its sub-steps, the ultrasonication treatment involves: ultrasonicating at a power of from about 100 W to about 600 W and a frequency of from about 10 kHz to about 50 kHz for about 2 s and pausing for about 2 s, for a duration of from about 1 h to about 4 h, so as to have an adequate intermolecular interaction forces of the components. In one embodiment, the ultrasonication power of the ultrasonication treatment is 20 W to 3000 W, further preferably 100 W to 1000 W; for example, the ultrasonication power may be 20 W, 50 W, 100 W, 150 W, 200 W, 300 W, 400 W, 500 W, 1000 W, 2000 W, 3000 W, etc. In one embodiment, the ultrasonication frequency of the ultrasonication treatment is 5 kHz to 100 kHz, further preferably 10 kHz to 50 kHz; for example, the ultrasonication frequency may be 5 kHz, 10 kHz, 20 kHz, 30 kHz, 40 kHz, 50 kHz, 60 kHz, 70 kHz, 80 kHz, 90 kHz, 100 kHz, etc. In one embodiment, the ultrasonication time of the ultrasonication treatment is 1 h to 4 h; for example, the ultrasonication time may be 1 h, 2 h, 3 h, 4 h, etc.

Preferably, in the above step (i) and its sub-steps, the temperature of the rotary evaporation is from about 60° C. to about 80° C.

### Skin Care Composition

The present disclosure is directed to a skin care composition comprising an effective amount of the argireline co-assembled system described above. The present disclosure is also directed to the use of the argireline co-assembled system described above for anti-wrinkle benefit. An embodiment of the present disclosure further provides a use of the argireline co-assembled system as described above in the preparation of a formulation product having effects such as moisturization and repairing, especially anti-wrinkle benefit.

In one embodiment, the mass proportion of the argireline co-assembled system in the formulation product is 0.5% to 10%, and the formulation product may be in the form of an anhydrous essence, an emulsion, a cream, an essence, a facial mask, or the like, without limitation.

The skin care compositions herein may be cosmetic compositions, pharmaceutical compositions, or cosmeceutical compositions, and may be provided in various product forms, including, but not limited to, solutions, suspensions, lotions, creams, gels, serums, essences, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion.

The compositions herein may be prepared by conventional methods of making topical skin care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

The composition can contain other peptides than argireline. Such other peptides can be present at a level of preferably from about 0.00001% to about 5%, more preferably from about 0.00005% to about 1%, still more preferably from about 0.0001% to about 0.5%, by weight of the composition.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Vitamin B₃ Compound

The compositions herein may optionally include a safe and effective amount of a vitamin B₃ compound. In some instances, the present compositions may contain 0.01% to 10%, by weight, of the vitamin B₃ compound, based on the weight or volume of the composition (e.g., 0.1% to 10%, 0.5% to 5%, or even 1% to %).

As used herein, “vitamin B₃ compound” means a compound having the formula:

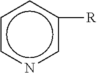

Where: R is CONH₂ (i.e., niacinamide), COOH (i.e., nicotinic acid) or CH₂OH (i.e., nicotinyl alcohol); derivatives thereof, and salts of any of the foregoing.

Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Dipeptide

The compositions herein may include a safe and effective amount of the palmitoylated diopeptide, pal-KT (INCI: Palmitoyl Dipeptide-7). In some instances, pal-KT may be present in the present compositions at 0.0001% to 3% (e.g., 0.0001% to 2%, 0.01% to 1% or 0.1% to 0.5%). Pal-KT is available as Palestrina® from Sederma (France).

### Tetrapeptide

The compositions herein may include a safe and effective amount of the acetylated tetrapeptide, ac-PPYL (INCI: Acetyl Tetrapeptide-11). In some instances, ac-PPYL may be present in the present composition at 0.0001% to 3% (e.g., 0.001% to 2%, 0.01% to 1% or 0.1% to 0.5%). Ac-PPYL is available as SYNIORAGE from BASF Care Creations (New Jersey).

### Pentapeptide

The compositions herein may include a safe and effective amount of the palmitoylated penta peptide, pal-KTTKS (INCI: Palmitoyl Pentapeptide-4). In some instances, pal-KTTKS may be present in the present compositions at 0.00005% to 3% (e.g., 0.00005% to 2%, 0.01% to 1% or 0.1% to 0.5%).

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

### Rheology Modifiers

The compositions herein may include 0.1% to 5% of a rheology modifier (e.g., thickening agent) to provide the composition with suitable rheological and skin feels properties. Some non-limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water:oil ratio (e.g., 12:1 to 1:1), and/or the ratio of water to thickener or oil to thickener.

### Emulsifiers

When the dermatologically acceptable carrier is in the form of an emulsion, it may be desirable to include an emulsifier to provide a stable composition (e.g., does not phase separate). When included, the emulsifier may be present at an amount of 0.1% to 10% (e.g., 1% to 5%, or 2%-4%). Emulsifiers may be nonionic, anionic or cationic. Some non-limiting examples of emulsifiers that may be suitable for use herein are disclosed in U.S. Pat. Nos. 3,755,560; 4,421,769; and McCutcheon's Detergents and Emulsifiers, North American Edition, pages 317-324 (1986).

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use of the Skin Care Composition

The present disclosure may also relate to a non-therapeutic method of treating skin, comprising:
a) identifying a target portion of skin where treatment is desired; and
b) applying the skin care composition of any preceding claim to the target portion of skin over the course of a treatment period.

The method improves penetration of the peptide derivative into skin.

The target portion of skin may be on a facial skin surface such as the forehead, perioral, chin, periorbital, nose, and/or cheek) or another part of the body (e.g., hands, arms, legs, back, chest). The person or target portion of skin in need of treatment may be one that exhibits a telltale sign of aging skin (e.g., fine lines, wrinkles, hyperpigmented spots). In some instances, a target portion of skin may not exhibit a sign of skin aging, but a user may still wish to treat the portion of skin if it is one that is known to exhibit visible signs of aging (e.g., skin that is exposed to the sun). In this way, the present methods and compositions may be used prophylactically to help delay the visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period herein is ideally of sufficient time for the composition to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### EXAMPLES

Examples of the present application will be described below. The examples described below are illustrative and only intended to explain the present application, and are not to be construed as limiting the present application. In the examples, where specific techniques or conditions are not specified, and techniques or conditions described in documents in the art are followed according to product instructions. The reagents or apparatuses used, for which no manufactures are specified, are all conventional products that are commercially available.

### Example 1

With reference to FIG. 1, specific steps of the method for preparing a betaine-malic acid deep eutectic solvent were as follows:

Before starting the preparation operation, a reaction vessel was first washed and sterilized with absolute ethanol. Under the protection of an inert gas, 1.0 equivalent weight of betaine, 1.0 equivalent weight of malic acid, and 1.0 equivalent weight of water were mixed. Stirring was conducted continuously at 60° C., 300 rpm for 12 h. Ultrasonication was performed at 300 W, 20 kHz for 2 s followed by pausing for 2 s, for a duration of 1 h, so as to increase the intermolecular interaction forces. After performing rotary evaporation to remove the water, the betaine-malic acid deep eutectic solvent was obtained.

### Comparative Example 2

Comparative Example 2 differed from Example 1 in that matrine was used to replace betaine as the ligand of malic acid. The synthesis steps were as follows:

Before starting the preparation operation, a reaction vessel was first washed and sterilized with absolute ethanol. Under the protection of an inert gas, 1.0 equivalent weight of matrine, 1.0 equivalent weight of malic acid, and 1.0 equivalent weight of water were mixed. Stirring was conducted continuously at 60° C., 300 rpm for 12 h. Ultrasonication was performed at 300 W, 20 kHz for 2 s followed by pausing for 2 s, for a duration of 1 h, so as to increase the intermolecular interaction forces. During rotary evaporation to remove the water, the precursor precipitated, and the deep eutectic solvent could not be formed.

### Comparative Example 3

Comparative Example 3 differed from Example 1 in that proline was used to replace betaine as the ligand of malic acid. The synthesis steps were as follows:

Before starting the preparation operation reaction vessel was first sterilized with absolute ethanol. Under the protection of an inert gas, 1.0 equivalent weight of proline, 1.0 equivalent weight of malic acid, and 1.0 equivalent weight of water were mixed. Stirring was conducted continuously at 60° C., 300 rpm for 12 h. Ultrasonication was performed at 300 W, 20 kHz for 2 s followed by pausing for 2 s, for a duration of 1 h, so as to increase the intermolecular interaction forces. After performing rotary evaporation to remove the water, the malic acid-proline deep eutectic solvent was obtained.

### Example 2

With reference to FIG. 1, specific steps of the method for preparing an argireline co-assembled system were as follows:

Under the protection of an inert gas, water and the betaine-malic acid deep eutectic solvent were added to a reaction kettle, stirring was performed continuously at 30° C., 300 rpm for 4 h, and high-pressure homogenization was performed at 6000 bar for 40 min such that the betaine-malic acid deep eutectic solvent was uniformly dispersed. Then, an argireline aqueous solution was added, wherein the mass ratio of argireline to betaine-malic acid was 1:25, and the mass fraction of argireline in the whole system was 0.2%. Then, an ultrasonic reactor was used to perform ultrasonication at 300 W, 20 kHz for 2 s followed by pausing for 2 s, for a duration of 1 h, so as to induce co-assembling of argireline and the betaine-malic acid deep eutectic solvent, thereby obtaining the argireline co-assembled system.

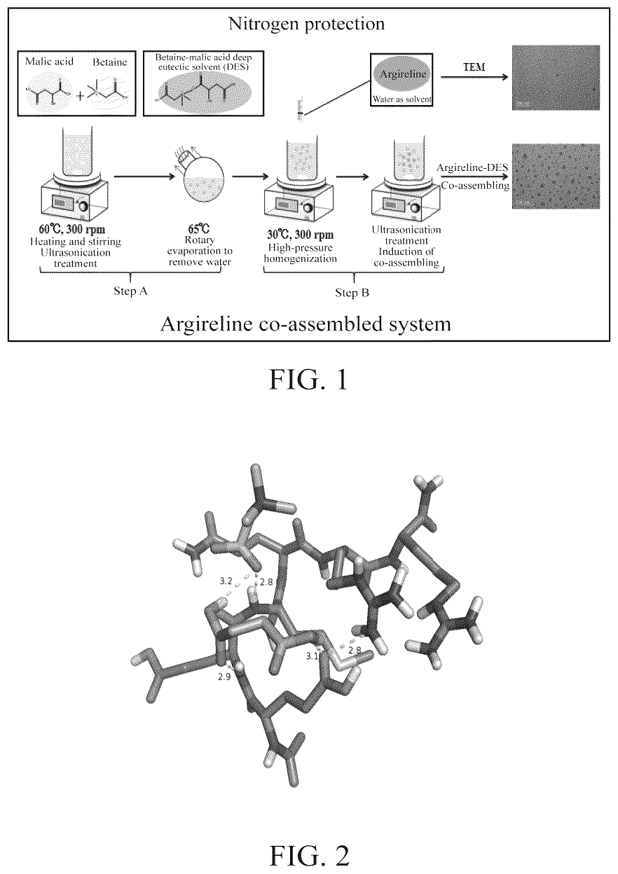

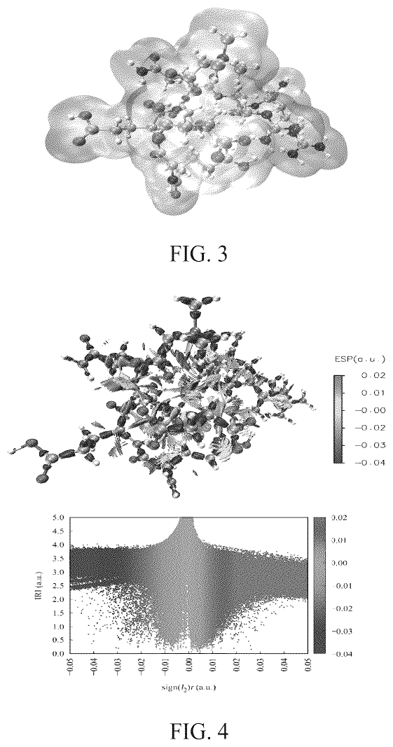

In order to investigate the sites of action between hydrogen bonds in the co-assembled system, molecular docking was performed with respect to argireline and the betaine-malic acid deep eutectic solvent. The most stable conformation was selected as a reference from the plurality of conformations produced, and only the hydrogen bonds which were most likely to occur were shown. As a result, with reference to FIG. 2, a total of five hydrogen bonds existed between the three molecules of argireline, betaine, and malic acid, among which one hydrogen bond existed between betaine and malic acid, the length of the hydrogen bond being 3.2 Å; one hydrogen bond existed between argireline and betaine, the length of the hydrogen bond being 2.8 Å; and three hydrogen bonds existed between argireline and malic acid, the lengths of the hydrogen bonds being 2.8 Å, 2.9 Å, and 3.1 Å, respectively. The result of the docking was used as the structure of the co-assembled system to calculate the molecular electrostatic potential. The electrostatic potential distribution is exemplified in FIG. 3, which provides a certain theoretical basis for subsequent use of the co-assembled system.

In addition, the interaction forces of the components of the argireline co-assembled system were calculated and analyzed. As exemplified in FIG. 4, besides the hydrogen bonds existing between the three components, various other non-covalent interaction, i.e., weak interactions existed within the co-assembled system, such as Van der Waals interaction, electrostatic interaction, etc., which promoted the three components to form the final co-assembled system through a co-assembling action.

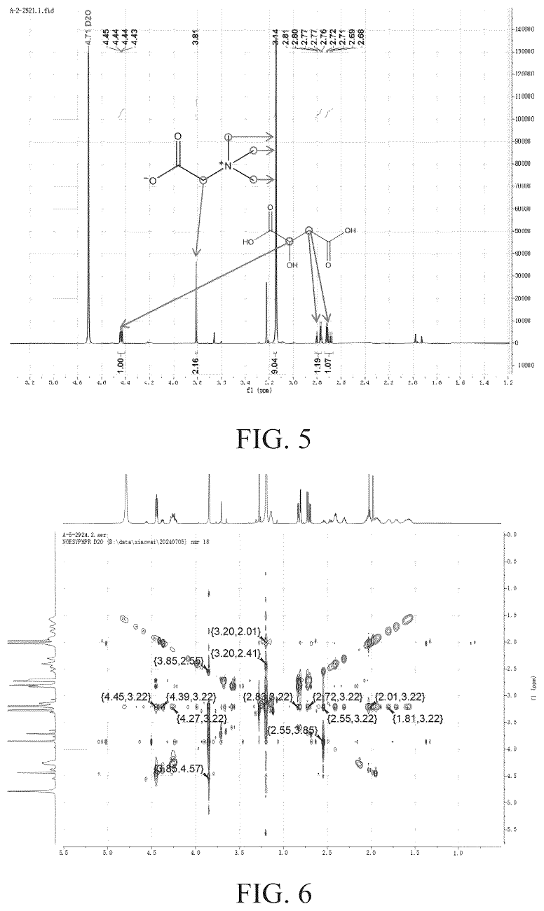

In order to verify whether betaine and malic acid in the betaine-malic acid deep eutectic solvent were still present in a ratio of 1:1, nuclear magnetic resonance hydrogen spectroscopy test was performed on the betaine-malic acid deep eutectic solvent prepared in Example 1. The nuclear magnetic resonance hydrogen spectrum is shown in FIG. 5. 1H NMR (500 MHz, Deuterium Oxide) δ 4.44 (dd, J=7.1, 4.5 Hz, 1H), 3.81 (s, 2H), 3.14 (s, 9H), 2.79 (dd, J=16.4, 4.5 Hz, 1H), 2.70 (dd, J=16.4, 7.1 Hz, 1H). The results of the chemical signals indicated that except for the active hydrogens in malic acid which were not shown, the characteristic peaks of both betaine and malic acid were present, and the ratio of the total number of hydrogen of betaine to that of malic acid was 11:3, i.e., the molar quantity ratio of betaine to malic acid was 1:1. That is, betaine and malic acid were still present in a ratio of 1:1 in the argireline co-assembled system.

At the same time, two-dimensional nuclear magnetic resonance hydrogen spectroscopy test was performed on the argireline co-assembled system prepared in Example 2. The two dimensional NMR spectrum is shown in FIG. 6: there were hydrogen-hydrogen correlation points between malic acid and betaine, which were, respectively, (4.45,3.22), (3.22, 4.45), (4.45-3.85), and (3.85-4.45); there were hydrogen-hydrogen correlation points between betaine and argireline, which were, respectively, (3.85,2.55), (2.55-3.85), etc.; and there were hydrogen-hydrogen correlation points between malic acid and argireline, which were, respectively, (4.45,1.94), (1.94-4.45), etc. It can be presumed that non-covalent interaction, i.e., weak interactions such as hydrogen bonds, van der Waals interactions, and electrostatic interactions might exist between any two of betaine, malic acid, and argireline, such that the three components were co-assembled to form a more compact supramolecular structure, resulting in an NOE effect and enabling observation of the hydrogen-hydrogen correlation points in the two dimensional nuclear magnetic spectrum.

### Comparative Example 4

Comparative Example 4 differed from Example 2 in that Comparative Example 4 involved an argireline aqueous solution without any treatment, and the mass fraction of argireline was 0.2%.

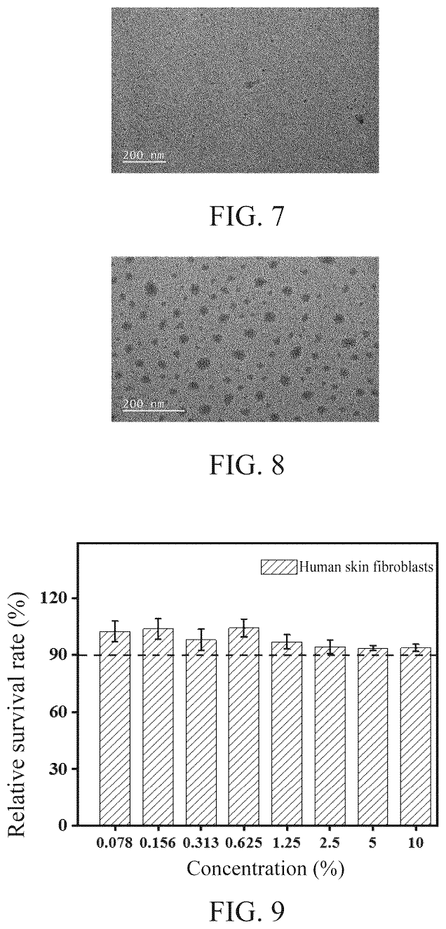

In order to enable more visualized observation of the co-assembling process and the details of the nanostructure formed so as to enable in-depth understanding of the self-assembly mechanism and optimization of the assembling process, transmission electron microscope (TEM) tests were performed on the argireline aqueous solution and the argireline co-assembled system, i.e., Comparative Example 4 and Example 2. It should be noted that, in the tests, the concentration of argireline in Comparative Example 4 and Example 2 was amplified synchronously. The test result of the argireline aqueous solution is shown in FIG. 7. The argireline aqueous solution at a certain concentration did not have a specific morphology. The phenomenon of aggregation of argireline could be observed in some parts of the field of vision, and the size of the aggregates varied. The test result of the argireline co-assembled system is shown in FIG. 8. Argireline and betaine-malic at certain concentrations could, after being induced by ultrasonication, co-assemble to form quasi-spherical assembled bodies. The particle size of the assembled bodies was around 33 nm, and were uniformly distributed in the field of vision.

### 1. Testing of the Biosafety of the Argireline Co-Assembled System Prepared in Example 2 Test Example 1

Whether a sample had a certain biological safety was determined by testing the cytotoxicity of the argireline co-assembled system on human skin fibroblasts.

Experimental principle: The viability of the cells was determined by using metabolic activity as an index. MTT (thiazolyl blue) is metabolically reduced in living cells to form blue-purple insoluble formazan, and the number of the living cells is correlated with the chromaticity determined photometrically after the formazan is dissolved in an alcohol.

Experimental method: The experiment was carried out according to SN/T2328-2009 “Keratinocyte cytotoxicity test on cosmetics acute toxicity”. Specific experimental procedure:
I: A suspension of human skin fibroblasts were seeded into a 96-well plate at 100 μL per well and incubated for 24 h.
II: After adherent culture of the cells for 24 hours, the culture medium was discarded, and 100 μL of a test sample solution was added per well. A blank group and a negative group were set.
III: After the cells were incubated and cultured for 24 h, 10 μL of MTT solution was added to each well, and the cells were cultured in an incubator for 4 h.
IV: The culture solution was discarded, 150 μL of DMSO was added to each well. After shaking for 10 min, the absorbance at 490 nm (OD_{490 nm}) was measured using a microplate reader. Comparison was made with the control sample not treated with the test substance, and the result was calculated.

$$Relative survival rate \% = \frac{Test {OD}_{490 n m}}{Neg {OD}_{490 n m}} * 100 \%$$

where:
Relative survival rate—Relative D_{490 nm}, %;
Test OD_{490 nm}−Average OD_{490 nm} of the test substance;
Neg OD_{490 nm}−Average OD_{490 nm} of the negative control.

A cytotoxicity test of the argireline co-assembled system was performed on human skin fibroblasts using the MTT model. The results of the test are shown in Table 1, and the trend of relative cell viability rate is shown in FIG. 9.

TABLE 1
Results of the action of the argireline co-assembled system on human skin fibroblasts

| | | Concentration by volume % | | | | | | | | | |
|---|---|---|---|---|---|---|---|---|---|---|---|
| | | NC | 0.078 | 0.156 | 0.313 | 0.625 | 1.25 | 2.5 | 5 | 10 | PC |
| Relative survival | | 100.00 | 102.43 | 103.74 | 97.97 | 104.29 | 96.91 | 94.30 | 93.67 | 93.72 | 31.11 |
| rate % | | | | | | | | | | | |
| SD % | | 4.62 | 5.52 | 5.78 | 5.46 | 4.86 | 3.65 | 3.48 | 1.31 | 1.80 | 0.81 |
| P-value | vs BC | 0.000 | / | / | / | / | / | / | / | / | / |
| | vs NC | / | 0.906 | 0.847 | 0.268 | 0.720 | 0.121 | 0.045 | 0.017 | 0.020 | 0.000 |

* Statistical method: A t-test approach was used for analysis, test level α = 0.05; P ≥ 0.05, indicating no statistical difference; 0.01 < P < 0.05, significant difference; P < 0.01, very significant difference; P < 0.001, extremely significant difference.

The experimental results are as shown in Table 1 and FIG. 9. When the argireline co-assembled system acted on human skin fibroblasts at concentrations within 10%, the cell relative survival rate was 90% or higher, showing good biocompatibility. That is, the argireline co-assembled system had good biosafety at concentrations within 10%.

2. Testing of Whether the Argireline Co-Assembled System Prepared in Example 2 would Cause Potential Adverse Responses in Human Skin.

### Test Example 2

Patch test method: A certified patch test device was selected, and an occlusive patch test method was used. 0.020 to 0.025 g of a test substance was placed in the patch test device, and was applied to the flexor side of the forearm of a subject by externally affixing the patch test device with a low sensitivity tape. After 24 hours, the test substance was removed, skin responses were observed at 0.5 h, 24 h, and 48 h after the removal, respectively. The results were recorded according to the skin response grading standard (Table 2) set forth in “Cosmetic Safety Technical Specification (2015 edition)”. The skin responses of the subject at different observation times are shown in Table 3.

TABLE 2
Skin response grading standard for skin occlusive patch test

| Response | Scoring | |
|---|---|---|
| degree | grade | Skin responses |
| − | 0 | Negative response |
| ± | 1 | Suspicious response, with only faint erythema |
| + | 2 | Weakly positive response (erythema response); Erythema, |
| | | infiltration, edema, possibly with papules |
| ++ | 3 | Strong positive response (herpes response); Erythema, infiltration, |
| | | edema, papules, herpes; The responses may extend out of the test |
| | | region. |
| +++ | 4 | Very strong positive response (confluent herpes response); |
| | | Significant erythema, severe infiltration, edema, confluent herpes; |
| | | The responses extend out of the test region. |

TABLE 3
Summary of the results of the human skin patch test

| | | | | Number of subjects in | | | | |
|---|---|---|---|---|---|---|---|---|
| | Number | | | different skin response | | | | |
| | of | Observation | | grades in the patch test | | | | |
| Group | Subjects | time | | 0 | 1 | 2 | 3 | 4 |
| Argireline co- | 31 | 0.5 | h | 31 | 0 | 0 | 0 | 0 |
| assembled | | 24 | h | 31 | 0 | 0 | 0 | 0 |
| system | | 48 | h | 31 | 0 | 0 | 0 | 0 |
| Negative control | 31 | 0.5 | h | 31 | 0 | 0 | 0 | 0 |
| Blank + filter | | 24 | h | 31 | 0 | 0 | 0 | 0 |
| disc | | 48 | h | 31 | 0 | 0 | 0 | 0 |

As can be seen from Table 3, no adverse responses occurred in the 31 subjects, indicating that the argireline co-assembled system had very low irritation and would not cause irritation to human skin. The argireline co-assembled system can be trustingly used in household chemical products.

### 3. Testing of the Anti-Wrinkle Effect of the Argireline Co-Assembled System Prepared in Example 2 and the Argireline Aqueous Solution Prepared in Comparative Example 4.

### Test Example 3

Whether or not a test sample had a firming and anti-wrinkle efficacy was evaluated by determining the change in the content of collagen type I in human skin fibroblasts after administration.

Experimental principle: Collagen type I is the main component of the extracellular matrix of the dermis, which is synthesized intracellularly and secreted extracellularly by dermal fibroblasts. Under the action of terminal procollagen peptidase, the terminal peptides are separated and polymerized to form collagen fibers. Therefore, the firming and anti-wrinkle efficacy of a test sample can be evaluated according to the content of collagen type I secreted by fibroblasts after administration.

Basis for test: Experiments were performed according to T/SHRH 031-2020 “Anti-wrinkly and Firming Lifting Efficacy Test of Cosmetics—In vitro Test Method of Collagen I Contents with Fibroblasts”.

Determination of results: A t-test approach was used for analysis, test level α=0.05; P≥0.05, indicating no statistical difference, and suggesting that the sample did not have an effect of promoting collagen type I secretion; P<0.05, indicating significant difference and up-regulation of the content of human collagen type I, suggesting that the sample had a firming and anti-wrinkle efficacy.

### Test Steps:

I (Cell plating): A suspension of human skin fibroblasts was seeded onto a 24-well plate, and the cells were grown adherently overnight.
II (Preparation): Working solutions of test substances were prepared respectively according to the test protocol (Table 4).

TABLE 4
Test protocol

| | | | Test method | | |
|---|---|---|---|---|---|
| Experimental | Sample information | | Test | Test | Test |
| group | Designation | Concentration | index | model | means |
| Blank control | / | / | Human | Human | ELISA |
| (BC) | | | collagen | skin | |
| Negative | / | / | type I | fibroblasts | |
| control (NC) | | | | | |
| Positive | TGF-β1 | / | | | |
| control (PC) | | | | | |
| Sample group | Argireline | 2.5% | | | |
| | aqueous solution | | | | |
| | Argireline co- | 2.5% | | | |
| | assembled system | | | | |

III (Administration): When the cells in the 24-well plate reached 40% to 60% confluence, grouped administration was performed according to the test protocol in Table 4. Each well was added with 250 μL of corresponding samples, and three replicate wells were set for each group. The samples were allowed to act for 48 h.
IV (ELISA Test): Supernatants were collected and tested according to the instructions of an ELISA kit.

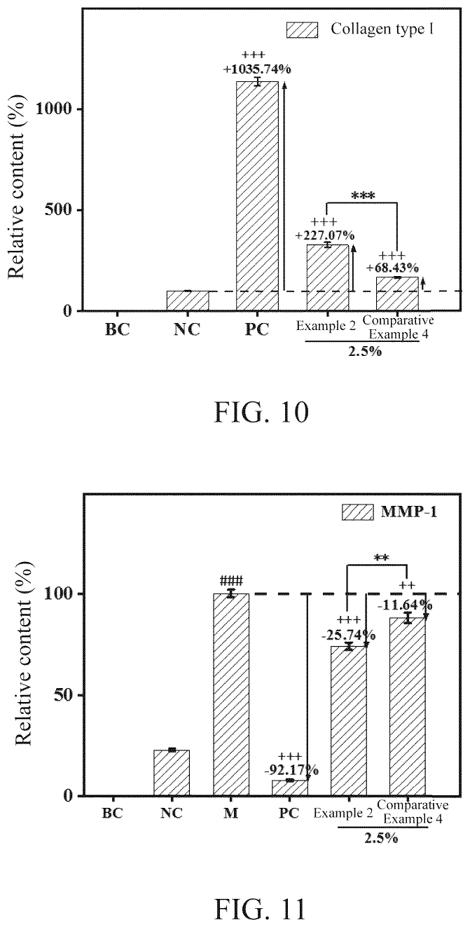

Test results: With the samples having been applied to human skin fibroblasts, the content of human collagen type I was determined by ELISA. The test results are shown in Table 5, and a comparison of the relative content of human collagen type I is shown in FIG. 10.

TABLE 5
Results of determination of the relative
content of human collagen type I

| | | Group | | | | |
|---|---|---|---|---|---|---|
| | | | | | | Argireline |
| | | | | | Argireline | co- |
| | | | | | aqueous | assembled |
| | | | | | solution | system |
| | | BC | NC | PC | (2.5%) | (2.5%) |
| Relative content % | | 0.00 | 100.00 | 1135.74 | 168.43 | 327.07 |
| SD % | | 0.00 | 2.63 | 21.32 | 3.81 | 13.02 |
| Up-regulation % | | / | / | 1035.74 | 68.43 | 227.07 |
| P-value | vs NC | / | / | / | 0.000 | 0.00 |
| | vs | / | / | / | / | 0.000 |
| | argireline | | | | | |
| | aqueous | | | | | |
| | solution | | | | | |
| | (2.5%) | | | | | |

As shown in Table 5 and FIG. 10, the content of human collagen type-I in the PC group was significantly increased compared with the NC group (P<0.05), indicating that this test was effective. Compared with the NC group, both the argireline aqueous solution and the argireline co-assembled system at 2.5% test concentration significantly increased the content of human collagen type I (P<0.05), the up-regulation rates being 68.43% and 227.07%, respectively. Hence, both the argireline aqueous solution and the argireline co-assembled system at 2.5% test concentration had the ability to promote the synthesis of human collagen type I and achieve a firming and anti-wrinkle efficacy.

In summary, at 2.5% concentration, the ability of the argireline co-assembled system to promote the synthesis of human collagen type I was better than that of the argireline aqueous solution (P<0.05), suggesting that at that concentration, the argireline co-assembled system had a better firming and anti-wrinkle efficacy than the argireline aqueous solution, approximately 3.3 times the firming and anti-wrinkle efficacy of the argireline aqueous solution.

### Comparative Test Example 1

Comparative Test Example 1 differed from Test Example 3 in that the anti-wrinkle efficacy of a sample was evaluated by determining the amount of MMP-1 released from human skin fibroblasts.

Experimental principle: MMP-1 is a member of the matrix metalloproteinase family. It can degrade various types of collagens and extracellular matrix molecules, and is an important factor leading to dermatolysis and occurrence of wrinkles. UV irradiation can induce MMP-1 secretion by fibroblasts in the corium layer. Determination of the amount of MMP-1 secreted by human skin fibroblasts can reflect the anti-wrinkle efficacy of a sample.

Basis for test: Experiments were performed according to PJJC-SOP3-65 “Standard Operation Procedures for the Determination of MMP-1 in Human Skin Fibroblasts”.

Determination of results: A t-test approach was used for analysis, test level α=0.05; P≥0.05, indicating no statistical difference, and suggesting that the sample did not have the effect of promoting a change in the content of MMP-1; P<0.05, indicating significant difference and a decrease in the content of MMP-1, suggesting that the sample had a firming and anti-wrinkle efficacy.

### Test Steps:

I (Cell Plating): A suspension of human skin fibroblasts was seeded onto a 24-well plate, and the cells were grown adherently overnight.
II (Preparation): Working solutions of test substances were prepared respectively according to the test protocol (Table 6).

TABLE 6
Test protocol

| | Sample information | | | | | |
|---|---|---|---|---|---|---|
| Experi- | | | Stimu- | Test method | | |
| mental | Desig- | Concen- | lation | Test | Test | Test |
| group | nation | tration | condition | index | model | means |
| Blank | / | / | / | MMP-1 | Human | ELISA |
| control | | | | | skin | |
| (BC) | | | | | fibro- | |
| Negative | / | / | / | | blasts | |
| control | | | | | | |
| (NC) | | | | | | |
| Model | / | / | UVA | | | |
| group | | | | | | |
| (M) | | | | | | |
| Positive | TGF-β1 | / | | | | |
| control | | | | | | |
| (PC) | | | | | | |
| Sample | Argireline | 2.5% | | | | |
| group | aqueous | | | | | |
| | solution | | | | | |
| | Argireline | 2.5% | | | | |
| | co- | | | | | |
| | assembled | | | | | |
| | System | | | | | |

III (Administration): UVA irradiation was performed at an irradiation dose of 4.8 J/cm². After completion of UVA irradiation, the medium in each well was replaced with a fresh, test substance-containing medium, and the 24-well plate was placed in cell culture for further culturing 48 h.
IV (ELISA Test): Supernatants were collected and tested according to the instructions of an ELISA kit.

Test results: With the samples having been applied to human skin fibroblasts, the content of MMP-1 was determined by ELISA. The test results are shown in Table 7, and a comparison of the relative content of MMP-1 is shown in FIG. 11.

TABLE 7
Results of determination of the relative content of MMP-1

| | | Group | | | | | |
|---|---|---|---|---|---|---|---|
| | | | | | | | Argireline |
| | | | | | | Argireline | co- |
| | | | | | | aqueous | assembled |
| | | | | | | solution | system |
| | | BC | NC | M | PC | (2.5%) | (2.5%) |
| Relative content % | | 0.00 | 23.18 | 100.00 | 7.83 | 88.36 | 74.26 |
| SD % | | 0.00 | 0.76 | 1.86 | 0.39 | 2.58 | 1.83 |
| Inhibition rate % | | / | / | / | 92.17 | 11.64 | 25.74 |
| P-value | vs NC | / | 0.000 | / | 0.000 | 0.003 | 0.000 |
| | vs argireline | / | / | / | / | / | 0.002 |
| | aqueous | | | | | | |
| | solution (2.5%) | | | | | | |

As shown in Table 7 and FIG. 11, the relative content of MMP-1 in the M group was significantly increased compared with the NC group (P<0.05), indicating that the stimulation condition of this test was effective. Compared with the M group, the relative content of MMP-1 in the PC group was significantly decreased (P<0.05), indicating that this test was effective. Compared with the M group, both the argireline aqueous solution and the argireline co-assembled system at 2.5% test concentration significantly decreased the relative content of MMP-1 (P<0.05), the inhibition rates being 11.64% and 25.74%, respectively. Hence, both the argireline aqueous solution and the argireline co-assembled system at 2.5% concentration could inhibit MMP-1 secretion and achieve an anti-wrinkle efficacy.

In summary, at 2.5% concentration, the ability of the argireline co-assembled system to inhibit MMP-1 secretion was better than that of the argireline aqueous solution (P<0.05), suggesting that at that concentration, the argireline co-assembled system had a better firming and anti-wrinkle efficacy than the argireline aqueous solution.

### Comparative Test Example 2

Comparative Test Example 1 differed from Test Example 3 in that the anti-wrinkle efficacy of a sample was evaluated by determining the amount of MMP-3 released from human skin fibroblasts.

Experimental principle: The degradation of extracellular matrix is mainly closely related to the metallomatrix protease MMP-3. When skin cells overexpress MMPS, the extracellular matrix in the corium layer is severely damaged, especially the normal structure of collagen fibers and elastic fibers. The anti-wrinkle efficacy of a test substance was evaluated by determining the content of matrix metalloproteinase-3 (MMP-3) in the extracellular matrix.

Basis for test: Experiments were performed according to PJJC-SOP3-65 “Standard Operation Procedures for the Determination of MMP-1 in Human Skin Fibroblasts”.

Determination of results: A t-test approach was used for analysis, test level α=0.05; P≥0.05, indicating no statistical difference, and suggesting that the sample did not have the effect of promoting a change in the content of MMP-3; P<0.05, indicating significant difference and a decrease in the content of MMP-3, suggesting that the sample had a firming and anti-wrinkle efficacy.

### Test Steps:

I (Cell Plating): A suspension of human skin fibroblasts was seeded onto a 24-well plate, and the cells were grown adherently overnight.

II (Preparation): Working solutions of test substances were prepared respectively according to the test protocol (Table 8).

TABLE 8
Test protocol

| | Sample information | | | | | |
|---|---|---|---|---|---|---|
| Experi- | | | Stimu- | Test method | | |
| mental | Desig- | Concen- | lation | Test | Test | Test |
| group | nation | tration | condition | index | model | means |
| Blank | / | / | / | MMP- | Human | ELISA |
| control | | | | 3 | skin | |
| (BC) | | | | | fibro- | |
| Negative | / | / | / | | blasts | |
| control | | | | | | |
| (NC) | | | | | | |
| Model | / | / | UVA | | | |
| group | | | | | | |
| (M) | | | | | | |
| Positive | TGF-β1 | / | | | | |
| control | | | | | | |
| (PC) | | | | | | |
| Sample | Argireline | 2.5% | | | | |
| group | aqueous | | | | | |
| | solution | | | | | |
| | Argireline | 2.5% | | | | |
| | co- | | | | | |
| | assembled | | | | | |
| | System | | | | | |

III (Administration): UVA irradiation was performed at an irradiation dose of 4.8 J/cm². After completion of UVA irradiation, the medium in each well was replaced with a fresh, test substance-containing medium, and the 24-well plate was placed in cell culture for further culturing 48 h.
IV (ELISA Test): Supernatants were collected and tested according to the instructions of an ELISA kit.

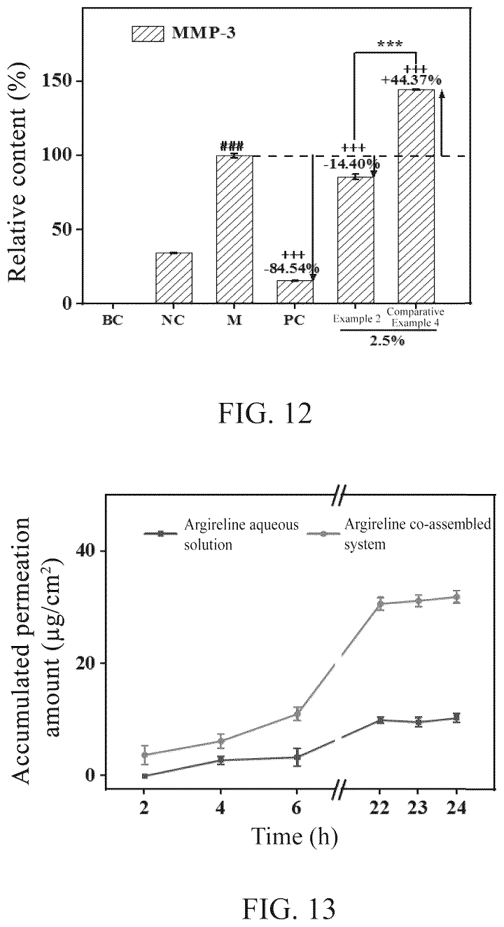

Test results: With the samples having been applied to human skin fibroblasts, the content of MMP-1 was determined by ELISA. The test results are shown in Table 9, and a comparison of the relative content of MMP-1 is shown in FIG. 12.

TABLE 9
Results of determination of the relative content of MMP-1

| | | Group | | | | | |
|---|---|---|---|---|---|---|---|
| | | | | | | | Argireline |
| | | | | | | Argireline | co- |
| | | | | | | aqueous | assembled |
| | | | | | | solution | system |
| | | BC | NC | M | PC | (2.5%) | (2.5%) |
| Relative content % | | 0.00 | 34.30 | 100.00 | 15.64 | 144.37 | 85.60 |
| SD % | | 0.00 | 0.36 | 1.49 | 0.19 | 0.55 | 1.76 |
| Inhibition rate % | | / | / | / | 84.54 | −44.37 | 14.40 |
| P-value | vs NC | / | 0.000 | / | 0.000 | 0.000 | 0.000 |
| | vs argireline | / | / | / | / | / | 0.001 |
| | aqueous | | | | | | |
| | solution (2.5%) | | | | | | |

As shown in Table 8 and FIG. 12, the relative content of MMP-3 in the M group was significantly increased compared with the NC group (P<0.05), indicating that the stimulation condition of this test was effective. Compared with the M group, the relative content of MMP-3 in the PC group was significantly decreased (P<0.05), indicating that this test was effective. Compared with the M group, the argireline co-assembled system at 2.5% test concentration significantly decreased the relative content of MMP-3 (P<0.05), the inhibition rate being 14.40%, Hence, the argireline co-assembled system at 2.5% concentration could inhibit MMP-3 secretion and achieve an anti-wrinkle efficacy. Compared with the M group, the argireline aqueous solution at 2.5% test concentration significantly decreased the relative content of MMP-3, suggesting that the argireline aqueous solution at 2.5% concentration did not have the effect of inhibiting MMP-3 secretion.

In summary, at 2.5% concentration, the argireline co-assembled system could inhibit MMP-3 secretion, showing a certain anti-wrinkle efficacy, whereas the argireline aqueous solution did not have an anti-wrinkle efficacy.

### 4. Testing of the In Vitro Transdermal Diffusion Effect of the Argireline Co-Assembled System Prepared in Example 2 and the Argireline Aqueous Solution Prepared in Comparative Example 4.

### Test Example 4

The skin is composed of stratum corneum, epidermis, dermis, subcutaneous tissue, etc. A drug will permeate into the skin after being placed on the surface of the skin. It will pass through the epidermis and reach the dermis. Due to the abundant capillaries in the dermis, the drug can be rapidly absorbed and then enter the systemic circulation. Therefore, the concentration of the drug at the surface of the skin is very low; that is, the so-called “sink” condition is met, and the concentration of the drug is close to 0. By means of an in vitro transdermal diffusion experiment, the transdermal absorption speed of a drug can be predicted, and the influence of a medium, a formulation composition, a transdermal absorption enhancer, etc. on the transdermal absorption speed of the drug can be investigated, which is a precondition for the effectiveness and safety of a transdermal preparation of the drug. Specific experimental method:
I (Microscopic examination): Pig skin without lesion was selected under an anatomical lens. Six pieces of skin having the same size were cut, rinsed with physiological saline once, and blotted with a filter paper to remove surface water.
II (Fixing skin): The skin was fixed on a Franz diffusion cell, with the stratum corneum facing the donor chamber and the corium layer facing the receptor chamber. 17 mL of physiological saline was added to the receptor chamber, and air bubbles were removed to ensure that there were no air bubbles between the corium layer and the receptor solution.
III (Administration): The instrument was switched on beforehand, and the water bath temperature was adjusted to 32±1° C. 1 mL of 2.5% argireline co-assembled system was added in the donor chamber. The sealing film was applied, and tin foil was added to prevent liquid evaporation. The effective permeation area was 0.367c cm².
IV (Permeation): The stirring speed was set to 300 rpm, and permeation was performed under light-proof condition.
V (Sampling): 1 mL of the liquid was taken from the receptor chamber at specific time points of 2 h, 4 h, 6 h, 22 h, 23 h, and 24 h, respectively, and filtered through an organic membrane having a pore size of 0.22 μm. Liquid phase determination was performed, and the cumulative permeation amount was calculated.

### Comparative Test Example 3

Comparative Test Example 3 differed from Test Example 4 in that the argireline co-assembled system at the administration step in Test Example 4 was replaced with an argireline aqueous solution.

Results of the test of cumulative permeation amount per unit area:

FIG. 13 is a statistical diagram of cumulative permeation amount per unit area of Test Example 4 and of Comparative Test Example 3 in an in vitro transdermal diffusion experiment. As shown in FIG. 13, at each time point of incubation with the skin, the cumulative permeation amount of the argireline co-assembled system in Test Example 4 was consistently much larger than that of the argireline aqueous solution in Comparative Test Example 3, exhibiting a more excellent transdermal effect. At 24 h of incubation with the skin, the cumulative permeation amount per unit area of the argireline co-assembled system was about 3.1 times that of the argireline aqueous solution.

### Skin Care Formulation Examples

The following are examples of skin care compositions which are suitably used as oil-in-water emulsion cream compositions.

These compositions can be made by any conventional methods, for example, by blending the A phase components with a suitable mixer (e.g., Tekmar RW20DZM or equivalent) and heating to a temperature of 50-80° C. and maintaining the temperature while stirring. Separately, the B phase components are blended with a suitable mixer and heated to 60-75° C., while maintaining temperature during mixing. Phase B is added to Phase A while mixing well to form an oil-in-water (O/W) emulsion. The emulsion is then milled using a suitable mill (e.g., Tekmar T-25 or equivalent) for 5 minutes. When the emulsion is at 45-60° C., phase C is added while continuing to mix. The emulsion is then milled for 5 minutes to provide a uniform composition.

| | % w/w | | | | | | |
|---|---|---|---|---|---|---|---|
| Component | A | B | C | D | E | F | G |
| Phase A | | | | | | | |
| Water Purified | QS | QS | QS | QS | QS | QS | QS |
| Panthenol | 1.00 | 0.50 | 1.00 | 1.00 | 1.00 | 1.00 | 0.50 |
| Glycerine 99% | 5.00 | 8.00 | 8.00 | 8.00 | 8.00 | 8.00 | 8.00 |
| Niacinamide | 5.00 | 0.00 | 5.00 | 5.00 | 5.00 | 5.00 | 5.00 |
| Pentylene glycol | 1.00 | 3.00 | 3.00 | 0.00 | 3.00 | 2.00 | 3.00 |
| Disodium EDTA | 0.05 | 0.05 | 0.05 | 0.05 | 0.05 | 0.05 | 0.05 |
| Polyacrylamide & | 2.00 | 1.0 | 3.5 | 3.5 | 2.5 | 3 | 2.00 |
| C13-14 Isoparaffin | | | | | | | |
| Phase B | | | | | | | |
| Cetyl Alcohol | 1.00 | 0.40 | 0.50 | 1.00 | 0.70 | 0.80 | 1.00 |
| Stearyl Alcohol | 1.25 | 0.50 | 1.00 | 1.25 | 0.80 | 1.00 | 1.20 |
| Behenyl Alcohol | 0.75 | 0.30 | 1.00 | 0.75 | 0.90 | 0.60 | 0.75 |
| PEG 100 Stearate | 0.10 | 0.10 | 0.10 | 0.10 | 0.10 | 0.20 | 0.20 |
| Cetearyl Glucoside | 0.20 | 0.20 | 0.20 | 0.20 | 0.20 | 0.20 | 0.40 |
| & Cetearyl alcohol | | | | | | | |
| Caprylic/Capric | 1.00 | 1.00 | 0.00 | 1.00 | 1.00 | 1.00 | 3.00 |
| Triglyceride | | | | | | | |
| Isohexadecane | | | | | 3.00 | 3.00 | 3.00 |
| Butyrospermum | 0 | 1.00 | 1.00 | 0 | 1.00 | 4.00 | 0 |
| Parkii (Shea Butter) | | | | | | | |
| Phase C | | | | | | | |
| Symsave H | 0.15 | | 0.15 | 0.15 | 0.15 | 0.00 | 0.15 |
| (preservative) | | | | | | | |
| Phenoxyethanol | 0.25 | 0.50 | 0.25 | 0.25 | 0.25 | 0.50 | 0.25 |
| Argireline co- | 0.01% | 0.05% | 0.5% | 1.25% | 2.5% | 5% | 10% |
| assemble system - | (mass | (mass | (mass | (mass | (mass | (mass | (mass |
| made by Ex. 2, but | ratio: | ratio: | ratio: | ratio: | ratio: | ratio: | ratio: |
| different mass ratio | 1:50) | 1:25 | 1:25 | 1:25 | 1:25 | 1:25 | 1:25 |
| of argireline to | | | | | | | |
| betaine-malic acid | | | | | | | |
| deep eutectic solvent | | | | | | | |

% w/w

| Component | H | I | J | K |
|---|---|---|---|---|
| Phase A | | | | |
| Water Purified | QS | QS | QS | QS |
| Panthenol | 0.50 | 0.50 | 0.50 | 0.50 |
| Glycerine 99% | 8.00 | 8.00 | 8.00 | 8.00 |
| Niacinamide | 5.00 | 5.00 | 5.00 | 5.00 |
| Pentylene glycol | 3.00 | 3.00 | 3.00 | 3.00 |
| Disodium EDTA | 0.05 | 0.05 | 0.05 | 0.05 |
| Polyacrylamide & | 2.50 | 2.50 | 2.00 | 2.00 |
| C13-14 Isoparaffin | | | | |
| Phase B | | | | |
| Cetyl Alcohol | 1.00 | 1.00 | 0.70 | 1.00 |
| Stearyl Alcohol | 1.25 | 1.25 | 0.80 | 1.20 |
| Behenyl Alcohol | 0.75 | 0.75 | 0.90 | 0.75 |
| Stearic acid and | | | | |
| Palmitic acid blend | | | | |
| (50/50) | | | | |
| PEG 100 Stearate | 0.10 | 0.10 | 0.10 | 0.10 |
| Cetearyl Glucoside | 0.20 | 0.20 | 0.20 | 0.20 |
| & Cetearyl alcohol | | | | |
| Isopropyl | | | | 1.33 |
| Isostearate | | | | |
| Isohexadecane | 3.00 | | 3.00 | 3.00 |
| Butyrospermum | | 1.00 | 2.00 | 3.00 |
| Parkii (Shea Butter) | | | | |
| Phase C | | | | |
| Symsave H | | | 0.15 | 0.15 |
| (preservative) | | | | |
| Phenoxyethanol | 0.50 | 0.50 | 0.25 | 0.25 |
| Argireline co- | 0.00001 | 0.00010 | 0.0010 | 0.01 |
| assemble system - | (mass | (mass | (mass | (mass |
| made by Ex. 2, but | ratio: | ratio: | ratio: | ratio: |
| different mass ratio | 1:50 | 1:75 | 1:25 | 1:50 |
| of argireline to | | | | |
| betaine-malic acid | | | | |
| deep eutectic | | | | |
| solvent | | | | |

Ex. A through Ex. K in below tables are the examples of the composition of the present disclosure. The examples of the composition of the present disclosure provide improved skin permeability of argireline and improved stability of argireline in skin care compositions, and are suitable for providing anti-wrinkle benefit.

### Example Combinations

Embodiment 1. An argireline co-assembled system, wherein the argireline co-assembled system is a co-assembled system formed from betaine-malic acid as a deep eutectic solvent and argireline through non-covalent interaction;
preferably, wherein the mass ratio of argireline to betaine-malic acid deep eutectic solvent is 1:5 to 1:100; and
preferably, wherein the molar quantity ratio of betaine to malic acid in the deep eutectic solvent is 1:5 to 5:1.

Embodiment 2. A method for preparing an argireline co-assembled system, the method comprising the steps of:
(i) preparing a deep eutectic solvent, the deep eutectic solvent being betaine-malic acid;
(ii) mixing argireline and the betaine-malic acid deep eutectic solvent and stirring at a temperature of from about 20° C. to about 40° C.;
(iii) performing ultrasonication to induce co-assembling of the argireline and the betaine-malic acid deep eutectic solvent, thereby obtaining the argireline co-assembled system; preferably, wherein the mass ratio of argireline to betaine-malic acid deep eutectic solvent is 1:5 to 1:100; and
preferably, wherein the molar quantity ratio of betaine to malic acid in the deep eutectic solvent is 1:5 to 5:1.

Embodiment 3. The method of Embodiments 2, wherein the above step (ii) mixing and stirring argireline and the betaine-malic acid deep eutectic solvent comprises the sub-steps of:
(ii-a) mixing water and the betaine-malic acid deep eutectic solvent, stirring at a temperature of from about 20° C. to about 40° C. and homogenizing at a pressure of from about 2000 bar to about 8000 bar, such that the betaine-malic acid deep eutectic solvent is uniformly dispersed;
(ii-b) mixing an argireline aqueous solution and the above uniform dispersion of betaine-malic acid deep eutectic solvent at a temperature of from about 20° C. to about 40° C.

Embodiment 4. The method of any of Embodiments 2 and 3, wherein the stirring time in the step (ii) is from about 1 h to about 12 h, and the stirring rotation speed in the step (ii) is from about 300 rpm to about 600 rpm.

Embodiment 5. The method of any of Embodiments 2-4, wherein the homogenization time in the step (ii) is from about 20 min to about 60 min.

Embodiment 6. The method of any of Embodiments 2-5, wherein the ultrasonication treatment in the step (iii) involves: ultrasonicating at a power of from about 100 W to about 600 W and a frequency of from about 10 kHz to about 50 kHz for about 2 s and pausing for about 2 s, for a duration of from about 1 h to about 4 h.

Embodiment 7. The method of any of Embodiments 2-6, wherein the step (i) the preparation of the betaine-malic acid deep eutectic solvent comprises the sub-steps of.
(i-a) under the protection of an inert gas, mixing betaine, malic acid, and an appropriate amount of water, stirring under heating,
(i-b) performing ultrasonication treatment, and
(i-c) performing rotary evaporation to remove water, thereby obtaining the betaine-malic acid deep eutectic solvent.

Embodiment 8. The method of any of Embodiments 2-7, wherein the inert gas in the step (i) is nitrogen, and the pressure of the nitrogen is 0.1 MPa to 20 MPa.

Embodiment 9. The method of any of Embodiments 2-8, wherein the stirring temperature in the step (i) is from about 30° C. to about 80° C., the stirring time in the step (i) is from about 1 h to about 48 h, and the stirring rotation speed in the step (i) is from about 300 rpm to about 600 rpm.

Embodiment 10. The method of any of Embodiments 2-9, wherein the ultrasonication treatment in the step (i) involves: ultrasonicating at a power of from about 100 W to about 600 W and a frequency of from about 10 kHz to about 50 kHz for about 2 s and pausing for about 2 s, for a duration of from about 1 h to about 4 h.

Embodiment 11. The method of any of Embodiments 2-10, wherein the temperature of the rotary evaporation in the step (i) is from about 60° C. to about 80° C.

Embodiment 12. The use of the argireline co-assembled system of any of the preceding Embodiments for anti-wrinkle benefit.

Embodiment 13. A skin care composition comprising an effective amount of the argireline co-assembled system of any of the preceding Embodiments.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An argireline co-assembled system, wherein the argireline co-assembled system is a co-assembled system formed from betaine-malic acid as a deep eutectic solvent and argireline through non-covalent interaction.

2. The argireline co-assembled system of claim 1, wherein the mass ratio of argireline to betaine-malic acid deep eutectic solvent is about 1:5 to about 1:100.

3. The argireline co-assembled system of claim 1, wherein the molar quantity ratio of betaine to malic acid in the deep eutectic solvent is about 1:5 to about 5:1.

4. A method for preparing an argireline co-assembled system, the method comprising the steps of:
(i) preparing a deep eutectic solvent, the deep eutectic solvent being betaine-malic acid;
(ii) mixing argireline and the betaine-malic acid deep eutectic solvent and stirring at a temperature of from about 20° C. to about 40° C.; and
(iii) performing ultrasonication to induce co-assembling of the argireline and the betaine-malic acid deep eutectic solvent, thereby obtaining the argireline co-assembled system.

5. The method of claim 4, wherein the mass ratio of argireline to betaine-malic acid deep eutectic solvent is 1:5 to 1:100.

6. The method of claim 4, wherein the molar quantity ratio of betaine to malic acid in the deep eutectic solvent is 1:5 to 5:1.

7. The method of claim 4, wherein the above step (ii) mixing and stirring argireline and the betaine-malic acid deep eutectic solvent comprises the sub-steps of:
(ii-a) mixing water and the betaine-malic acid deep eutectic solvent, stirring at a temperature of from about 20° C. to about 40° C. and homogenizing at a pressure of from about 2000 bar to about 8000 bar, such that the betaine-malic acid deep eutectic solvent is uniformly dispersed;
(ii-b) mixing an argireline aqueous solution and the above uniform dispersion of betaine-malic acid deep eutectic solvent at a temperature of from about 20° C. to about 40° C.

8. The method of claim 7, wherein the stirring time in the step (ii) is from about 1 h to about 12 h, and the stirring rotation speed in the step (ii) is from about 300 rpm to about 600 rpm.

9. The method of claim 7, wherein the homogenization time in the step (ii) is from about 20 min to about 60 min.

10. The method of claim 4, wherein the ultrasonication treatment in the step (iii) involves: ultrasonicating at a power of from about 100 W to about 600 W and a frequency of from about 10 kHz to about 50 kHz for about 2 s and pausing for about 2 s, for a duration of from about 1 h to about 4 h.

11. The method of claim 4, wherein the step (i) the preparation of the betaine-malic acid deep eutectic solvent comprises the sub-steps of:
(i-a) under the protection of an inert gas, mixing betaine, malic acid, and an appropriate amount of water, stirring under heating,
(i-b) performing ultrasonication treatment, and
(i-c) performing rotary evaporation to remove water, thereby obtaining the betaine-malic acid deep eutectic solvent.

12. The method of claim 11, wherein the inert gas in the step (i) is nitrogen, and the pressure of the nitrogen is about 0.1 MPa to about 20 MPa.

13. The method of claim 11, wherein the stirring temperature in the step (i) is from about 30° C. to about 80° C., the stirring time in the step (i) is from about 1 h to about 48 h, and the stirring rotation speed in the step (i) is from about 300 rpm to about 600 rpm.

14. The method of claim 11, wherein the ultrasonication treatment in the step (i) involves: ultrasonicating at a power of from about 100 W to about 600 W and a frequency of from about 10 kHz to about 50 kHz for about 2 s and pausing for about 2 s, for a duration of from about 1 h to about 4 h.

15. The method of claim 11, wherein the temperature of the rotary evaporation in the step (i) is from about 60° C. to about 80° C.

16. The use of the argireline co-assembled system of claim 1 for anti-wrinkle benefit.

17. A skin care composition comprising an effective amount of the argireline co-assembled system of any of claim 1.
